(19) United States
(12) Patent Application Publication
(10) Pub. No.: US 2011/0226703 A1
(43) Pub. Date: Sep. 22, 2011

(54) METHOD FOR STERILIZING LIQUID AND LIQUID STERILIZATION DEVICE

(75) Inventors: Ron Kemmer, Wertheim (DE); Berthold Conradt, Wiesbaden (DE)

(73) Assignee: BRITA GMBH, TAUNUSSTEIN (DE)

(21) Appl. No.: 12/998,778

(22) PCT Filed: Dec. 1, 2009
(86) PCT No.: PCT/EP2009/066157

§ 371 Date: May 31, 2011

(30) Foreign Application Priority Data
Dec. 2, 2008 (DE) 10 2008 044 292.5

(51) Int. Cl. C02F 1/32; A61L 2/10; F25D 3/00

(52) U.S. Cl. 210/739; 210/748.11; 210/138; 62/389

(57) ABSTRACT

A method for sterilizing liquids, wherein quantities of liquid flowing through at least one reaction chamber are irradiated by at least one UV lamp and are removed after irradiation. The UV lamp continues to be operated for a period Δt₁ after removal of the quantity of liquid. A liquid sterilization device comprising at least one UV lamp and at least one reaction chamber through which the liquid to be sterilized flows as well as a tap for removing quantities of liquid. A control and/or regulation device is connected to the tap and to the UV lamp and continues to operate the UV lamp for a period Δt₁ once the tap is switched off.

### FIELD OF THE INVENTION

The invention relates to a method for sterilizing liquids, wherein portions of liquid flowing through at least one reaction chamber are irradiated by at least one UV lamp and removed after irradiation. The invention also relates to a liquid sterilization device.

### BACKGROUND OF THE INVENTION

Water sterilization devices are also integrated into refrigeration appliances in order to sterilize cooled water delivered by the refrigeration appliance. In this context, it is essential that the cooled water is not warmed up by the sterilization device. On the other hand, the cold water flowing through the reaction chamber cools the surroundings of the reaction chamber, thus also cooling the UV lamp which is located near the reaction chamber. As long as the UV lamp is in operation, however, the UV lamp will not be cooled to an extent adversely affecting its function.

The water is usually removed in portions from such devices, e.g. to fill drinking glasses one after the other. Once a particular removal act has finished, cooled water remains in the reaction chamber in order to be instantly available for the next dispensing act. The volume of water corresponding to the volume of the reaction chamber cools, amongst others, the UV lamp if the latter is only operated during the removal act. As a result, the lamp temperature falls quite rapidly, depending on the water temperature, so that in case of another dispensing act commencing shortly afterwards the UV lamp is either not ready for use or it takes too long until the UV output required for sterilization is provided.

If there are longer breaks between the dispensing or removal acts, this problem usually does not occur since the UV lamp, which initially cooled down, will gradually warm up to ambient temperature, i.e. as a rule to room temperature (RT=20° C.), as does the cooled water in the reaction chamber, and as a result the UV lamp will usually have its starting temperature when another dispensing act is then initiated.

The UV lamp is at its starting temperature if the UV lamp is able to reach its intended UV output within 30 seconds after having been switched on. The starting temperature of the UV lamp usually lies within the range of from 15° C. to 20° C.

### SUMMARY OF THE INVENTION

It is therefore an object of the invention to provide a method for sterilising liquids where the UV lamp has at least its starting temperature at the beginning of each liquid removal act, without the need to operate the UV lamp continuously, which would lead to unnecessary power consumption.

This object is achieved by means of a method in which liquid at a temperature below the starting temperature of the UV lamp is sterilised, the UV lamp continues to be operated for a period Δt₁ after removal of a portion of liquid, and the period Δt₁ is selected such that the volume of liquid retained in the reaction chamber is warmed up at least to the starting temperature of the UV lamp.

It has been found that the UV lamp need not be operated during the entire break between two removal acts, which would lead to unnecessary power consumption since the same quantity of liquid would be irradiated all the time.

It is sufficient to continue to operate the UV lamp only for a defined period Δt₁ after removal of a portion of water, i.e. after the dispensing process.

This period Δt₁ is selected such that the volume of liquid remaining in the reaction chamber is preferably warmed up such that the UV lamp is prevented from permanently cooling down to below its starting temperature. It is therefore preferred that the period Δt₁ be selected such that the volume of liquid retained in the reaction chamber is warmed up at least to the starting temperature of the UV lamp.

Since this starting temperature is usually within the range of the room temperature, it is sufficient to select the period Δt₁ such that the volume of liquid retained in the reaction chamber is warmed up at least to room temperature.

The liquid which is sterilised is preferably water. The liquid can have a temperature below the starting temperature of the UV lamp and/or below room temperature (RT=20° C.). Preferably, liquids at a temperature of particularly ≦12° C., particularly preferred ≦7° C., are sterilised. As another embodiment, water containing additives can be sterilised.

It is therefore preferred that the lower the temperature of the liquid to be sterilised is, the longer the period the Δt₁ is selected to be.

It is sufficient to continue to operate the UV lamp for a period Δt₁ if the starting temperature of the lamp approximately corresponds to the ambient temperature of the liquid sterilization device.

In the case that the ambient temperature of the liquid sterilization device should be below room temperature (RT=20° C.) or below the starting temperature of the UV lamp and the breaks between the removal acts are quite long, the UV lamp will cool down to ambient temperature over time. In these cases, it is preferred that the UV lamp be operated for at least a further period Δt₂ between the removal of two portions of liquid.

In the case of long breaks between the removal processes, it is recommended to operate the UV lamp for a period Δt₂ again and again, to ensure that the UV lamp has at least its starting temperature at the beginning of each subsequent removal process.

In this context, it is advantageous if the lamp temperature is measured and at least the action of switching on the UV lamp for operation of the UV lamp for the period Δt₂ is carried out in dependence on the lamp temperature.

The length of the period Δt₁ and/or of the period Δt₂ can also be regulated in dependence on the lamp temperature that has been measured. This method has the advantage that the number of periods Δt₂ as well as the lengths of the periods Δt₁ and/or Δt₂ can be suitably adapted in the case of changing ambient conditions.

In this way, it is ensured that the UV lamp is always ready for operation while power consumption is minimal.

The object is achieved by means of a liquid sterilization device which is characterised in that a liquid at a temperature below the starting temperature of the UV lamp flows through the reaction chamber (4), that a switching and/or control device is provided which is connected to the dispensing device and to the UV lamp, wherein the switching device continues to operate the UV lamp for a period Δt₁ after the switching off of the dispensing device, wherein the period Δt₁ is selected such that the volume of liquid retained in the reaction chamber after the dispensing device has been switched off is warmed up to at least the starting temperature of the UV lamp.

According to another embodiment, the switching device is configured such that it continues to operate the UV lamp at least for a further period Δt₂ between the removal of two portions of liquid.

Preferably, at least one temperature sensor is provided. The temperature sensor is preferably arranged at the UV lamp for measuring the lamp temperature.

This temperature sensor or a further temperature sensor can also be a bimetallic element for measuring the ambient temperature. The bimetallic element is preferably arranged in the switching device and/or control device. If, however, the switching and/or control device itself generates much heat, it is preferred that the bimetallic element be provided outside the switching and/or control device.

Preferably, the volume of the reaction chamber<the volume of a portion of liquid. This has the advantage that the volume of liquid which is located in the reaction chamber during longer idle times and is warmed up to room temperature there makes only a small contribution to the total volume of the portion of liquid during the next act of dispensing cooled water. As an overall result, a cooled portion of liquid can be provided in this manner.

The liquid sterilization device is preferably used in a refrigeration appliance or in conjunction with an appliance for dispensing cooled liquid, in particular cooled water.

### BRIEF DESCRIPTION OF THE DRAWING

The invention will be better understood and other features and advantages will become apparent by reading the detailed description of the invention, taken together with the drawing 1, wherein:

FIG. 1 is a schematic representation of a liquid sterilization device.

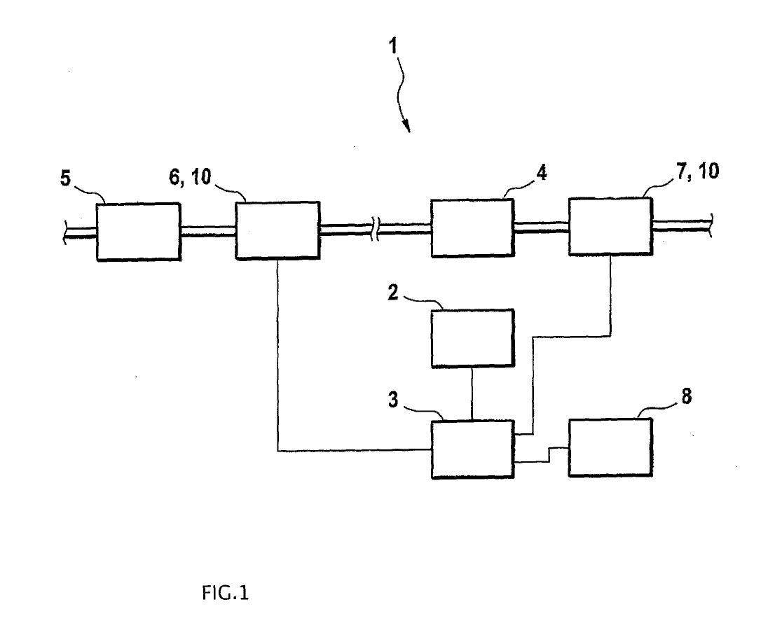

### DETAILED DESCRIPTION OF THE INVENTION

In the accompanying FIGURE, a liquid sterilization device 1 is schematically depicted, which can be connected to a water pipe by means of a connector 5. The water supplied flows through a valve 6 and enters a reaction chamber 4, where the water is irradiated by a UV lamp 2, thus sterilizing it. The sterilised water leaves the water sterilization device 1 via an outlet 7. The outlet 7 may be provided with a push button or the like to start the dispensing process.

The valve 6 and the outlet 7 are parts of the dispensing device 10, which are connected to a switching and/or control device 3. To initiate the dispensing process, the push button at the outlet 7 is actuated, whereupon the switching and/or control device 3 opens the valve 6 to allow water from the water pipe to flow in via the connector 5. Once the dispensing act has finished, the valve 6 is closed by the switching and/or control device 3.

Furthermore, a temperature sensor 8 is provided which is also connected to the switching and/or control device 3. Once the dispensing act has finished, the switching and/or control device continues to operate the UV lamp 2 for the period Δt₁. The switching and/or control device 3 illustrated in the FIGURE is also designed to operate the UV lamp 2 at least for a further period Δt₂ between two removals of portions of liquid.

### LIST OF REFERENCE NUMERALS

1 Liquid sterilization device
2 UV lamp
3 Switching and/or control device
4 Reaction chamber
5 Connector
6 Valve
7 Outlet
8 Temperature sensor
10 Dispensing device

What is claimed is:

1. A method of sterilizing liquids, comprising the steps of: irradiating portions of liquid flowing through at least one reaction chamber by at least one UV lamp having a starting temperature from 15° C. to 20° C. and removing portions of liquid from the reaction chamber after irradiation, wherein liquid at a temperature below the starting temperature of the UV lamp is sterilized, the temperature of the liquid being less than or equal to 12° C., continuing to operate the UV lamp for a period Δt₁ after removal of a portion of liquid, and
selecting the period Δt₁ such that the volume of liquid retained in the reaction chamber is warmed up at least to the starting temperature of the UV lamp.

2. The method according to claim 1, wherein the lower the temperature of the liquid to be sterilized is, the longer the period Δt₁ is selected to be.

3. (canceled)

4. The method according to claim 1, wherein the UV lamp is operated for at least a further period Δt₂ between the removal of two portions of liquid.

5. The method according to claim 1, wherein the lamp temperature is measured and that at least the action of switching on the UV lamp for operation of the UV lamp during the period Δt₂ is carried out in dependence on the lamp temperature.

6. The method according to claim 1, wherein the lamp temperature is measured and the length of the period Δt₁ is regulated in dependence on the temperature of the UV lamp.

7. The method according to claim 1, wherein the liquid that is sterilized is water or water containing additives.

8. A liquid sterilization device, comprising: at least one UV lamp having a starting temperature from 15° C. to 20° C., and comprising at least one reaction chamber through which the liquid flows, the temperature of the liquid being less than or equal to 12° C., and comprising a dispensing device for removing portions of liquid, wherein liquid at a temperature below the starting temperature of the UV lamp flows through the reaction chamber, wherein
wherein a switching and/or control device is provided which is connected to the dispensing device and to the UV lamp, and that the switching and/or control device continues to operate the UV lamp for a period Δt₁ after the switching off of the dispensing device, wherein the period Δt₁ is selected such that the volume of liquid retained in the reaction chamber after the dispensing device has been switched off is warmed up to at least the starting temperature of the UV lamp.

9. The device according to claim 8, wherein the switching and/or control device continues to operate the UV lamp at least for a period Δt₂ between the removal of two portions of liquid.

10. The device according to claim 8, wherein at least one temperature sensor is provided.

11. The device according to claim 10, wherein the temperature sensor is arranged at the UV lamp for measuring the lamp temperature.

12. The device according to claim 10, wherein the temperature sensor is a bimetallic element for measuring the ambient temperature.

13. The device according to claim 12, wherein the bimetallic element is arranged in the switching and/or control device.

14. (canceled)

15. A refrigeration appliance and/or an appliance for dispensing cooled liquid comprising the liquid sterilization device according to claim 8.

16. The method according to claim 1, wherein the volume of the reaction chamber is selected to be smaller than the volume of the portion of liquid.

17. The method according to claim 2, wherein the UV lamp is operated for at least a further period Δt₂ between the removal of two portions of liquid.

18. The method according to claim 17, wherein the lamp temperature is measured and that at least the action of switching on the UV lamp for operation of the UV lamp during the period Δt₂ is carried out in dependence on the lamp temperature, and wherein the lamp temperature is measured and at least one of the length of the period Δt₁ and the length of the period Δt₂ is regulated in dependence on the temperature of the UV lamp.

19. The method according to claim 18, wherein the liquid that is sterilized is water or water containing additives.

20. The device according to claim 9, wherein at least one temperature sensor is provided.

21. The device according to claim 20, wherein the temperature sensor is arranged at the UV lamp for measuring the lamp temperature.

22. The device according to claim 20, wherein the temperature sensor is a bimetallic element for measuring the ambient temperature.

23. The method according to claim 4, wherein the lamp temperature is measured and the length of the period Δt₂ is regulated in dependence on the temperature of the UV lamp.
